(19) United States
(12) Patent Application Publication
(10) Pub. No.: US 2004/0128078 A1
(43) Pub. Date: Jul. 1, 2004

(54) System, method and article of manufacture for flexibly setting measurements in a scalable tracking system

(76) Inventors: John F. Haughton, Winchester, MA (US); J. Anthony Jenkens, Winchester, MA (US)

Correspondence Address: MAUREEN STRETCH, ATTORNEY AT LAW, 26 CHARLES STREET, NATICK, MA, 01760, US

(21) Appl. No.: 10/334,498

(22) Filed: Dec. 31, 2002

(51) Int. Cl. G06F 17/60; G06F 19/00; G01N 33/48; A61B 5/00; G06F 17/00; G01N 33/50; G06F 7/00

(52) U.S. Cl. 702/19; 600/300; 705/2; 707/104.1

(57) ABSTRACT

A system for flexibly setting measurements in a tracking system having the ability to track data about a subject so that the measurements serve as the goals for any schedules, examinations, and tests for evaluating the subjects being tracked.

### BACKGROUND

1. Technical Field

This invention relates generally to the field of tracking information and more particularly to systems and methods for flexibly setting measurement goals and targets in a scalable tracking system.

2. Background

On Mar. 29, 1999, a Boston, Mass. physician, Dr. John Noble, of the Boston University School of Medicine, was reported as saying that outcome tracking against performance measures was “haphazard” for most health care practices.

Present patient tracking or registry systems do not provide health care entities and providers with an easy to use, extensible system for setting, tracking and modifying care guidelines and clinical goal measures in a flexible manner.

### DRAWINGS

FIG. 1 is a schematic diagram of the invention.

FIG. 2 is a table illustrating an embodiment of the invention.

FIG. 3 is a schematic drawing of a screen display showing an aspect of the invention.

FIG. 4 is a schematic drawing of a screen display showing an aspect of the invention.

FIG. 5 is a schematic drawing of a screen display showing an aspect of the invention.

FIG. 6 is a schematic drawing of an embodiment of the invention.

FIG. 7 is a flow diagram of the invention.

FIG. 8 is a schematic drawing of a screen display showing an aspect of the invention.

FIG. 9 is a schematic drawing of a screen display showing an aspect of the invention.

FIG. 10 is a schematic drawing of a screen display showing an aspect of the invention.

FIG. 11 is a schematic drawing of a screen display showing an aspect of the invention.

FIG. 12 is a block diagram of an aspect of the invention.

FIG. 13 is a schematic drawing of a screen display showing an aspect of the invention.

FIG. 14 is a schematic drawing of a screen display showing an aspect of the invention.

FIG. 15 is a schematic drawing of a screen display showing a report provided by the invention.

FIG. 16 is a schematic drawing of a screen display showing a report provided by the invention.

FIG. 17 is a schematic drawing of a screen display showing a report provided by the invention.

FIG. 18 is a schematic drawing illustrative tables used by an embodiment of the invention.

FIG. 19 is a block diagram showing a list of tables used in an embodiment of the invention.

FIG. 20 is a flow diagram of the invention.

FIG. 21 is a schematic diagram of a screen display showing a report provided in an embodiment of the invention.

FIG. 22 is a block diagram of a table showing another aspect of the invention.

### DETAILED DESCRIPTION OF THE INVENTION

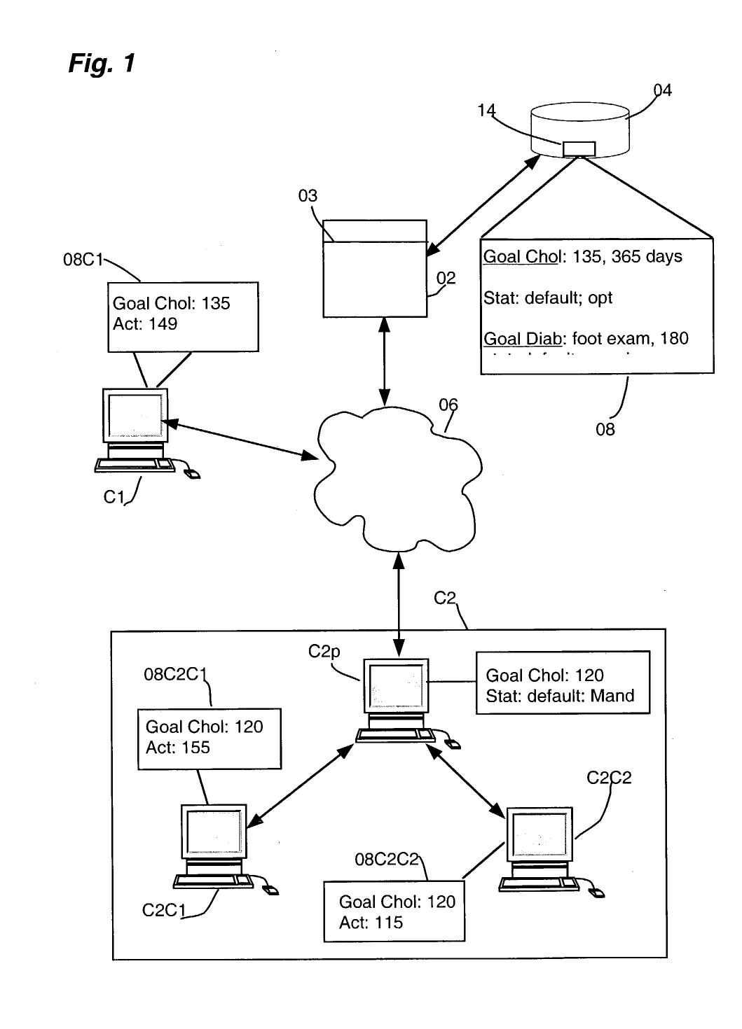

In FIG. 1, an overview of an embodiment of the present invention is shown. Parent system 02 with setting function 03 of the present invention is shown in communication with the internet 06, and through the internet 06, or some other communication medium, various “child” systems, C1 and C2 in a familial relationship. Parent system 02 also communicates with a database 04, which contains a global set of measurements for a plurality of conditions. While the present invention was developed primarily for evidence-based medicine for health care and health related conditions, those skilled in the art will appreciate that it can also be applied in other situations in which companies, organizations, or other entities wish to set goals based on evidence or best practices or objectives and to measure actual results for each subject against target goals, practices, objectives, etc. for a number of items.

Advances in scientific studies have led to the development of evidence-based medicine. As more scientific studies demonstrate the efficacy of various treatments and measures in improving or maintaining health (the evidence) more practitioners and insurers are interested in applying these treatments and measures in regular, daily practice. One difficulty arises in simply becoming aware of the number and variety of evidence-based best practices or goals for the variety of medical conditions a health care practitioner may encounter. Another difficulty is remembering when and how to apply them and what the details of use are. A third area of difficulty involves tracking the effects of various conditions over time as they relate to a particular patient or group of patients.

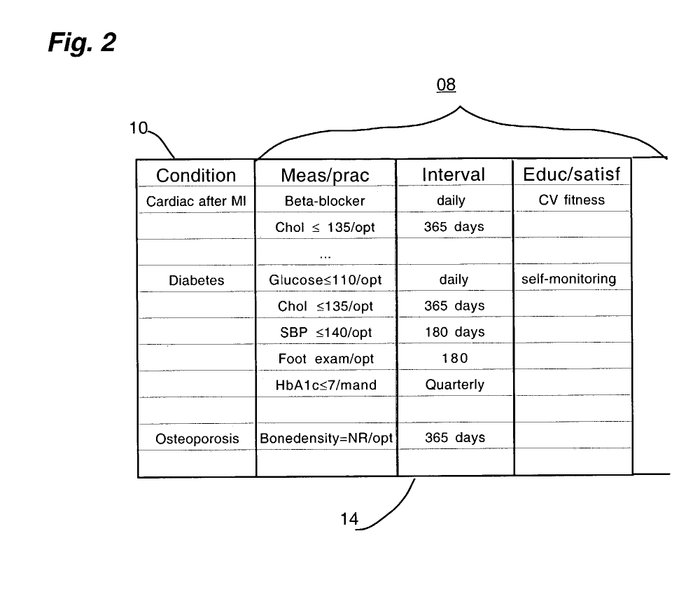

The present invention addresses these points in a longitudinal patient encounter, outreach, and profiling tracking registry—parent system 02 of FIG. 1, which enables an entity to create a master set of measurements 14 in a global data area, which for health care, contain conditions 10 and corresponding measures 08, as illustrated in FIG. 2. As can be seen in FIG. 2, one condition 10, such as Diabetes, may have many measures 08 related to it. For example, clinical studies have shown that self-monitoring of blood sugar daily by a patient and Hemoglobin A1C quarterly or semi-annually by the healthcare system are extremely effective measures of diabetic control and care. Other studies have shown that frequent foot exams by a health care professional can help prevent some of the unfortunate complications that can arise from diabetes because some patients ignore small blisters or cuts until life-threatening complications set in.

Similarly, and referring to FIG. 2, other studies have shown that beta-blockers, prescribed for daily use after a myocardial infarction tend to reduce significantly the likelihood of a second heart attack or other complications. With the present invention, this best practice can be included as one of the measures 08, which are linked to a cardiac after MI condition 10. In the prior art, a general practice physician was usually left to his or her own devices to remember to consider medications in this manner. One study showed that only about 20% of physicians in that study remembered to prescribe beta-blockers for patients with recent myocardial infarctions.

A difficulty faced by the health care profession is that there are so many conditions which may require treatment or watching, so much administrative information related to procedures, formularies, etc., and so much information to absorb and use, that it is difficult to keep up with the information and even more difficult in some cases to remember when and how to apply it. In other professions and industries, there are analogous problems. Anytime a complex subject can be measured or analyzed in a variety of ways for a variety of factors, implementing best practices or evidence-based measurements to use as goals or targets against which results are measured can be difficult.

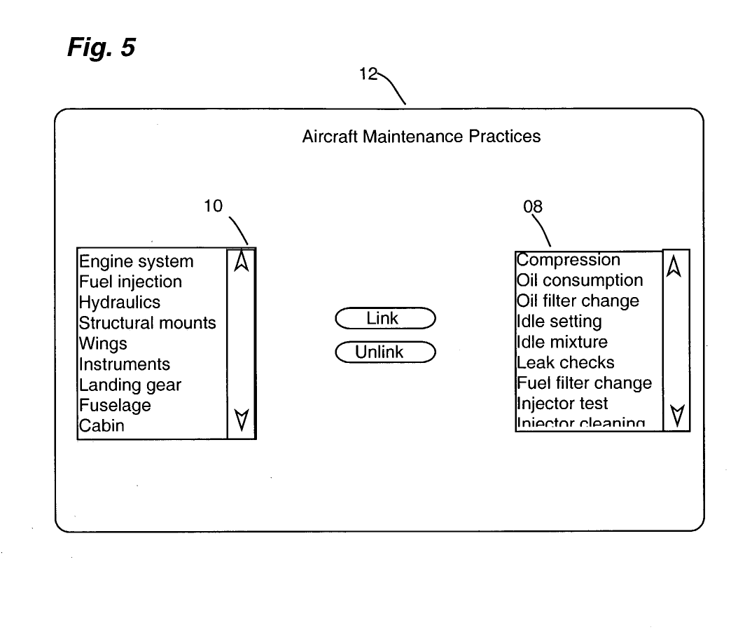

Now in FIG. 2, in the embodiments shown, the present invention includes a master set of measurements 14 with conditions 10 and measures 08, which are of use in a general health care practice. In the embodiments shown, conditions 10 are selected to correspond with those listed in the International Classification of Diseases, Ninth Revision, Clinical Modification (ICD-9-CM). Those skilled in the art will appreciate that all of the conditions 10 listed therein or only a subset of them may be used in the master set of measurements 14. In the embodiments shown, the master set of measurements 14 includes links between conditions 10 and measures 08 as part of the global measurement set up. That is, the master set of measurements 14 includes measures 08 or conditions 10 and may include sets of both conditions 10 and measures 08. Similarly, different kinds of master sets of measurements 14 could be used without deviating from the spirit of the invention. This is illustrated in FIG. 5, where conditions 10 as shown in screen display 12 are the main elements of an aircraft which need to be evaluated over time for maintenance purposes and the measures 08 contain various target values and recommended intervals for doing maintenance on the aircraft.

Those skilled in the art will appreciate that a master set of measurements 14, can be any set of best practices or goals and objectives that can be used to evaluate complex states or conditions in a subject, such as health in a human or animal, or the maintenance status of a fleet of airplanes, automobiles, or ships. Similarly, while the embodiments shown store the master set of measurements 14 as linked groups of conditions 10 and measures 08, those skilled in the art will appreciate that other arrangements are possible, such as a three or four (or more) tiered structure, or even a single tier structure containing a long list of potentially applicable measurements. In a health care system, for example, the master set of measurements 14 might also include prescription formularies from different insurers. In a manufacturing best practices system, master set of measurements 14 might include such things as failure rates for components, assembly time, costs to assemble, etc. which have been derived from best in class benchmark studies.

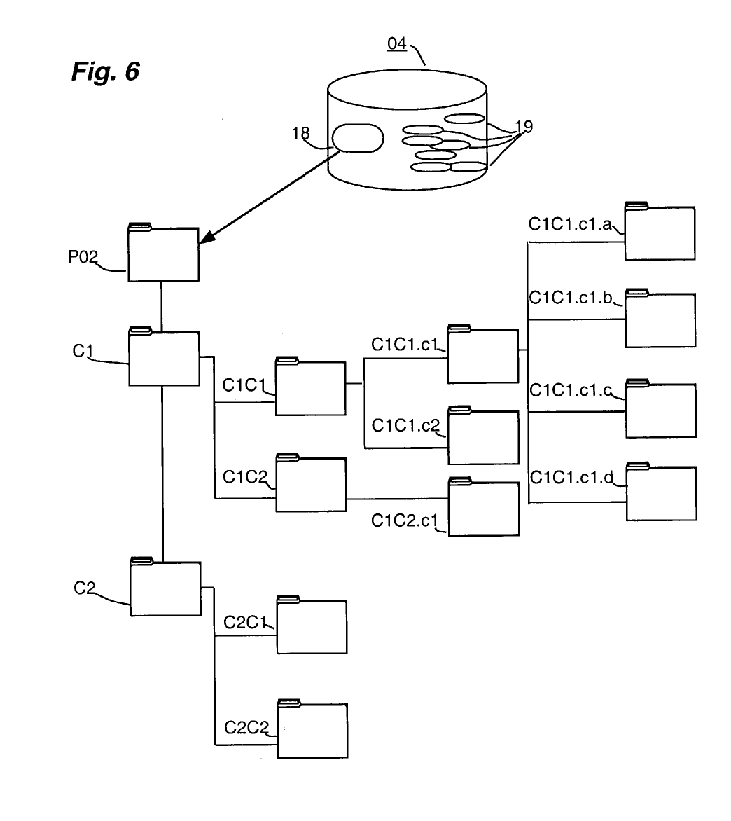

The present invention enables a plurality of users to share the same master set of measurements 14 across multiple sites, as shown in FIG. 6. In FIG. 6, parent system 02 is shown having two direct “children” C1 and C2. All three of these—parent system 02, child C1 and child C2—could be at different physical locations across the country, connected by the internet 06 or other network, or they could be three systems located in the same health care facility, such as a major hospital with several clinics communicating over an internal network or intranet, or even as terminals of a single mainframe computer. Each child C, may have multiple children, grandchildren, and so on, as also shown in FIG. 6. As is described in more detail below, the present invention also allows these shared measures to be set and changed in a scalable fashion.

In the embodiments shown, and still in FIG. 6, an administrator at the parent system 02 site would establish the master set of measurements 14, in global data 18 in database 04. As parent system 02 adds child sites, the master set of measurements 14 in global data 18 are propagated to site specific data 19 for each child site, as will be explained in more detail below. Setting function 03 of the present invention enables administrators to add or modify the master set of measurements 14 for their sites and child sites.

Still in FIG. 6, a user at child C2 may want to modify some of the measurements 14 for use at child site C2, and all its progeny. For example, if parent system 02 is a major hospital system with a number of affiliated clinics, child C2 might be a special cardiac outpatient clinic that is piloting some stricter guidelines for general care of cardiac outpatients. If the master set of measurements 14 includes a target goal of 135 mg for a cholesterol value, based on standard evidence-based practices, child C2 may want to set a target of 100, using setting function 03 based on results that might have come from a more recent study. If the parent system 02 allows this value to be modified (as will be explained in more detail below), child C2 may do so and propagate the new target value to its children.

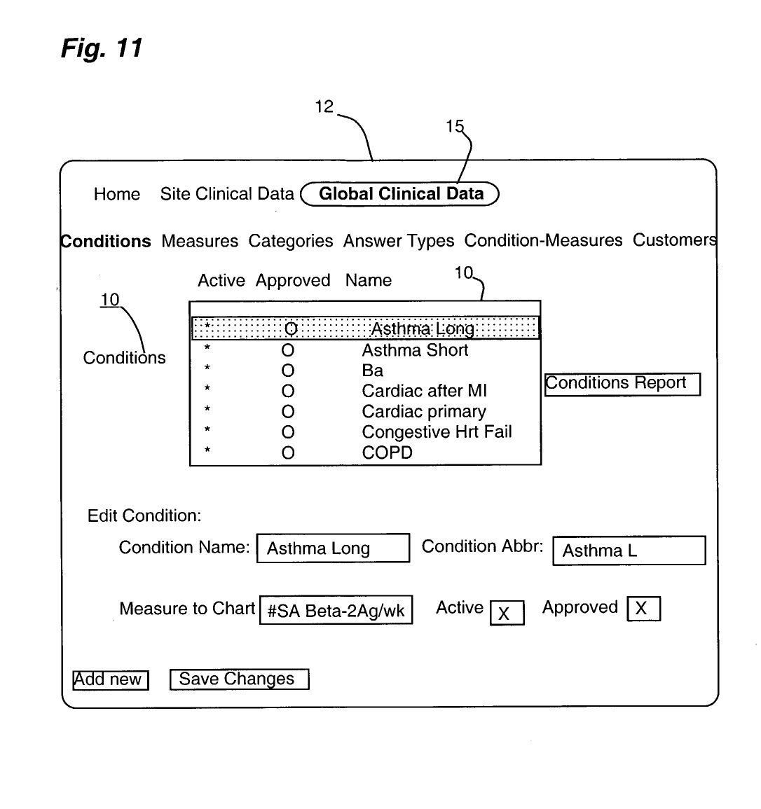

With reference to FIG. 11, a screen display 12 for global clinical data 15 for conditions 10 is shown. As sites are created by the system, users at each site are assigned security levels. When the present invention is implemented as a web based application service, the host employees of the web application service provider and the highest level users at each customer site are given global administrator security levels, which authorizes them to establish or modify global clinical data 15. In the embodiments shown, screen display 12 in FIG. 11 for global clinical data for conditions 10 will only be available to those users having such a security level. As shown in FIG. 11, an administrator at this highest level can add new conditions 10 or edit existing ones using setting function 03.

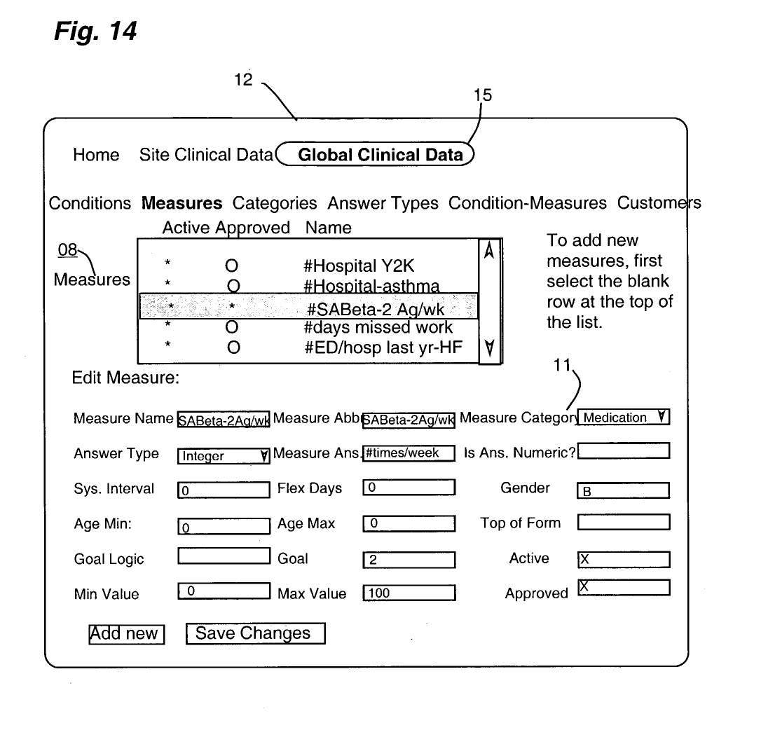

In FIG. 14, a screen display 12 for global clinical data 15 for measures 08 is shown. As can be seen, in the embodiments shown, there are a number of aspects of measures 08 which can be specified, such as type, frequency, applicability to gender, and so on. Those skilled in the art will appreciate that aspects such as these might be quite different for other uses of the present invention, such as aircraft maintenance, manufacturing best practices, etc.

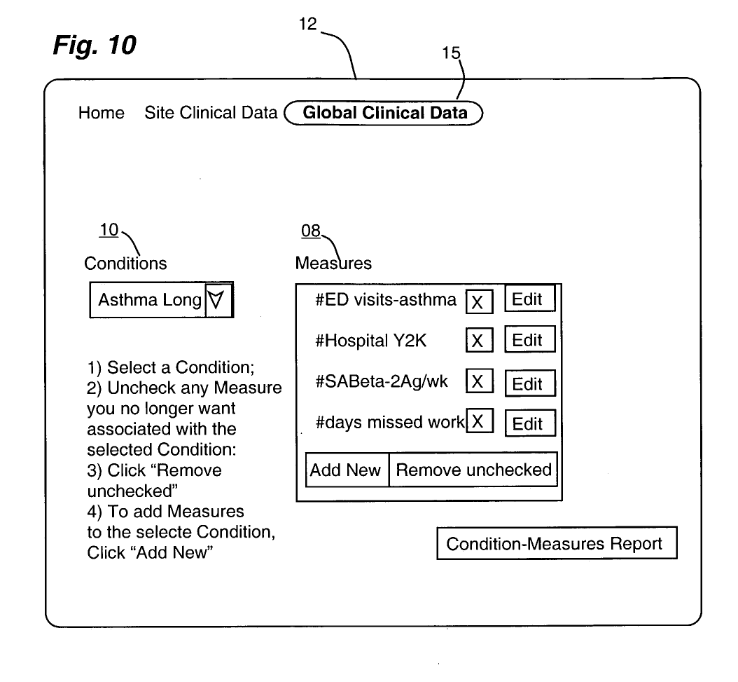

FIG. 10 shows a screen display 12 which a system administrator might use to edit linked conditions 10 and measures 08 in global clinical data 15.

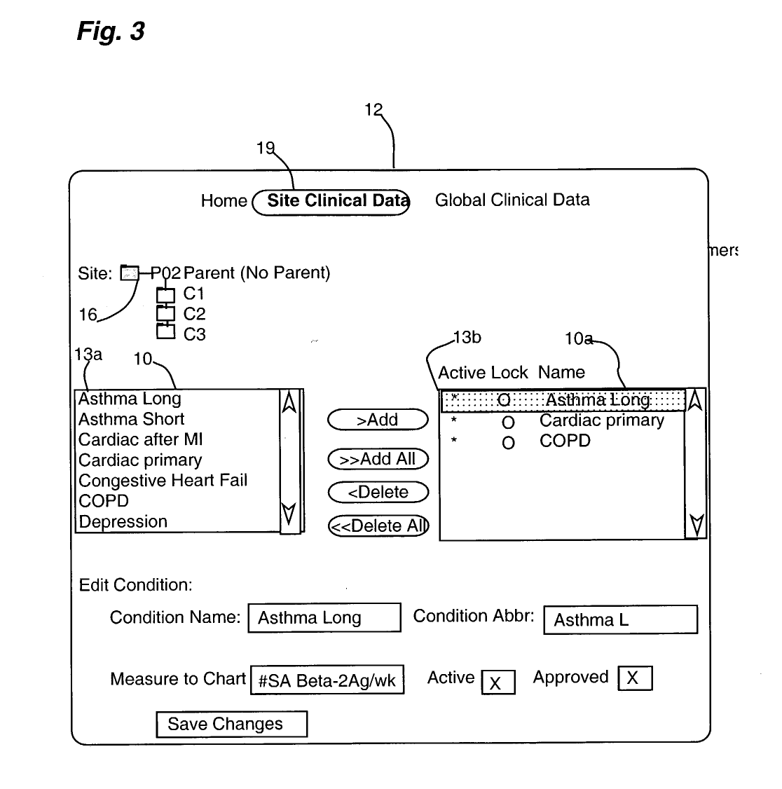

Now referring to FIG. 3, a screen display 12 for site clinical data 19 for conditions 10 is seen. In the embodiments shown, a site administrator may have access to site clinical data 19 for its own site and any subordinate or child sites below it. The present invention provides a tree control 16 for site data screen displays 12, which shows the top node (P02 in this instance) as the logged in user's site—the rest of the nodes in tree control 16 are that site's subordinate or child sites (in this example, C1-C3).

Still in FIG. 3, the approved conditions 10 from the parent site, which may be active or inactive at the parent site, fill listbox 13a. The right listbox, 13b fills with approved conditions 10a for this site and its children, based on the site selected in tree control 16. A site administrator can add or edit conditions 10 for its own site or its subordinate or child sites. When adding conditions 10 to the site, the present invention in the embodiments shown will automatically add all measures 08, categories 11, and condition-measure links 10-08. If the condition 10 being added is inactive at the parent level it will be made active at the current site, unless the condition has been locked at the parent site and therefore cannot be changed at the child site.

Still in FIG. 3, a site administrator may choose to edit a condition 10 for its site and/or some or all of its subordinate or child sites, if that condition 10 has not been locked at a higher level site.

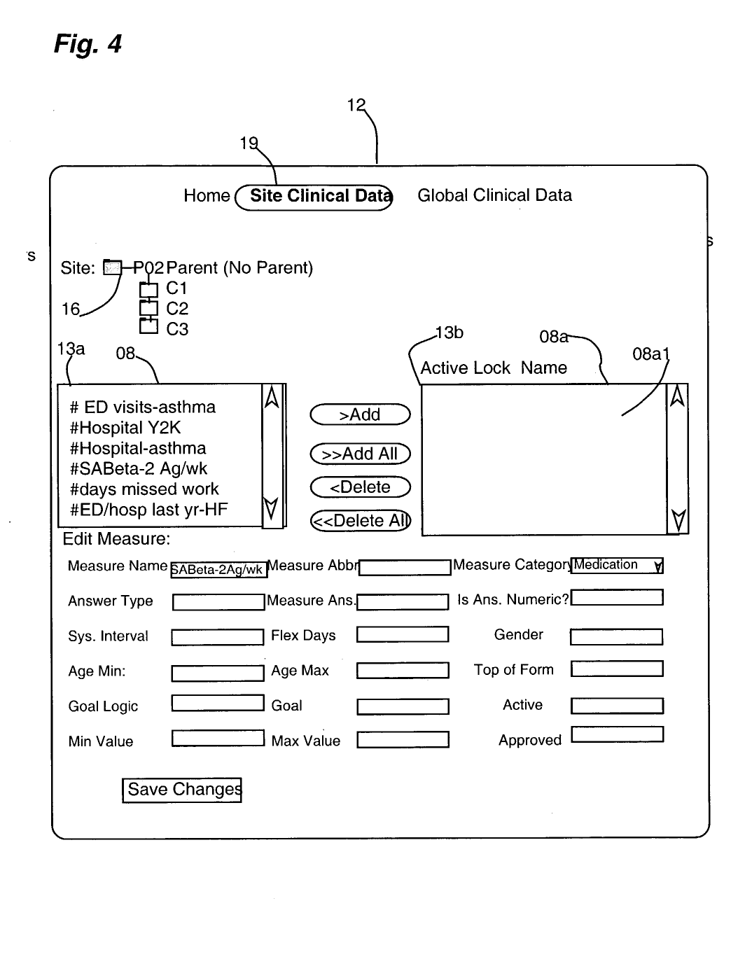

Turning now to FIG. 4, a screen display 12 showing site clinical data 19 for measures 08 is shown. In this example, a screen display 12 for editing site clinical data for measures 08 is provided. In much the same way as the site clinical data 19 for conditions is handled, a measure 08, such as #SABeta2-Ag/wk cannot be edited if it has been locked at a higher level site. A measure such as number of emergency department visits-asthma 08a1, is not locked in this example and thus could be edited by the site administrator to set a different goal value or to set other measure 08 properties.

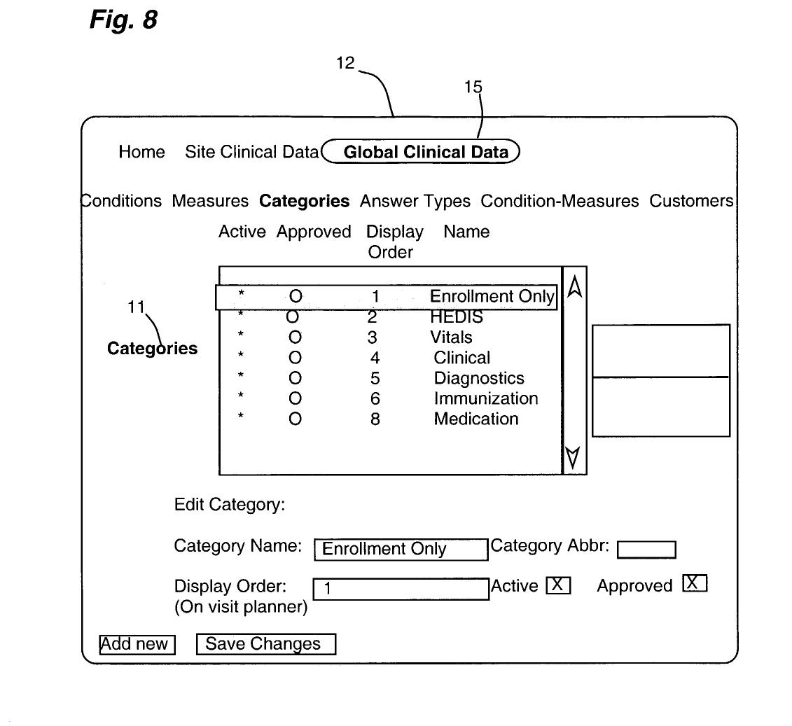

In the embodiments shown, master set of measurements 14 is standardized into categories 11 and answer types 5, so that they can be analyzed and compared properly. For example, and turning briefly to FIG. 8, categories 11 for health care might include clinical data, diagnostic data, immunization data, medication, and so on. Categories 11 help the user to display information in a familiar manner. They do not alter the underlying data structure or the relationship between the measures 08 and conditions 10. Categories 11 organize measures 08 for a condition 10 or groups of measures 08 related to more than one condition 10 into logical groups for display (reading) and data entry (writing). They allow the user experience to include user-customized groups of measures 08 for display and data entry. In the embodiments shown, a condition 10 has one or more measures 08. A category 11, likewise, can have one or more measure 08. The organization of measures 08 in categories 11 is independent of the organization of measures 08 in conditions 10. A measure 08 is typically in one and only one category 11.

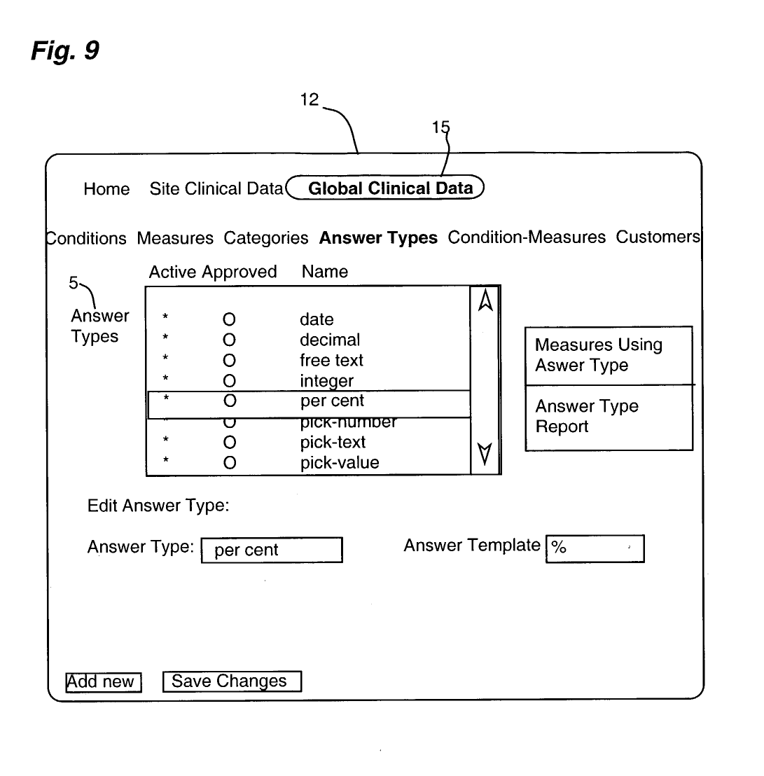

Next, in FIG. 9, values that can be entered into the present invention are also classified by answer type 5. To illustrate, a cholesterol value might have an integer answer type 5, while a prescription for a beta-blocker might have a pick-text answer type 5, in which the user has to pick from pre-defined text, such as “prescribe beta-blocker.” In some cases a value may be a date or a time interval, such as 365 days for annually scheduled events, or 180 days for exams to be done every 6 months. Other values might be classified as percentages and so on. Typically, the parent system 02 will define these answer types 5 for all measurements 14 and make all of them non-modifiable, to insure that analysis can be done on the same basis from site to site, even if the values themselves are modified by some of the sites. For example, an integer value may be required for the answer type for cholesterol testing, but the user at parent system 02 may allow its children to modify the target value for their sites, as long as it remains an integer value.

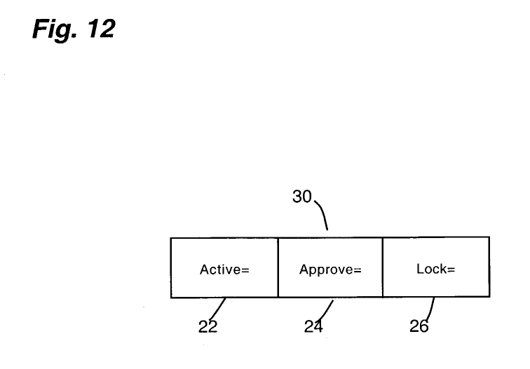

Turning now to FIG. 12, in the embodiments shown, all master sets of measurements 14 have settings for each of the indicators 30 associated with them. Thus the present invention provides a setting function 03 that enables a user to change site specific data in accordance with the indicators associated with the master set of measurements 14 and to scale those settings appropriately. For example, a measure 08 will have a setting for active 22, approve 24 and lock 26. So measure 08 for cholesterol may have active 22 equal to yes, approve 24 equal to yes and lock 26 equal to no. Each of the indicators 30 has a special function. The approve 24 indicator 30, means that this measurement 14, whether it is a condition 10 or a measure 08, in the embodiments shown, is approved for use by all in the system.

Indicators 30 make it easier for a user to customize the set of measurements 14. For example, a test that was once considered to be useful may be shown by recent evidence to be both inaccurate and wasteful, thus its status can be changed from an active test to an inactive one without deleting the information stored about the now inactive test. Additionally, the active 22 flag can be used to control how a measure is shared among sites. For example, a hospital may administer and house the database for multiple clinics. An orthopedic clinic may want a measure about bone pain to show up on its visit planner, while a peer or subordinate site dealing with vascular conditions may not want a bone pain measure. The indicator 30 for active 22 set to yes permits a measure 08 to be used at one site, but not another.

Similarly, a measurement 14 which had originally been marked active 22=yes, might be changed to active 22=no, to discourage use of a practice that has been found to be problematic. Conversely, a new test might be included in the master set of measurements 14, but initially marked active but not approved, until more research confirms its utility for global use to the administrators of parent system 02. The test could be not approved at a parent level, but active 22 could be set to true, in which case a subordinate child clinic could have the measure 08 active 22 (active 22 set to true or yes), so that one of the clinics in the system can be evaluating the test, yet since it is not approved, it cannot be used outside of the familial relationship which created it.

The approve 24 indicator 30 is used in the embodiments shown to permit users to share their modifications with others outside of the immediate familial relationship. In conjunction with the security controls provided by the system, it also controls unbridled dissemination without any process or order. In the example cited above of the cardiac clinic child C2 using a different value for the cholesterol measure 08, this might have initially been marked as approve=no, which would restrict its visibility to cardiac clinic C2 and its child sites. After some months have passed, the clinic might learn that using this modified target value has produced much better results for them than the normal target does. As a result, the users at the cardiac clinic child C2 may discuss sharing this goal and its results with others in the system. If the administrator at parent system 02 agrees that this may be a valuable modification, indicator 30 for approve 24 may be set to yes. Once the measure approval flag approve 24 is set to yes, all other sites in the system have access to the measure authored by C2. Without approve 24 being set to yes for this measure 08, C2 and its subordinates can use the measure 08, but other sites do not have access to that measure 08.

Also in the embodiments shown, a lock 26 indicator 30 is included. When lock 26 is marked yes, or on, then the item in the master set of measurements 14 cannot be changed by anyone except a user administrator with the authority to change the lock 26 indicator 30. Locking a measurement, in effect, makes it mandatory for that user system and its children. Thus, a user at parent system 02 may mark cholesterol as unlocked, using lock 26 indicator 30, but the user with appropriate security privileges at cardiac clinic child C2 may modify the value as described above and then change the status to lock 26 equal to yes for itself and its children, to insure that all the cardiac clinic sites use the same target value.

Another example of the use of lock 26 is with patient satisfaction measure(s) 08. A hospital or a health plan may want to collect patient satisfaction data in a consistent way throughout the organization. By locking the satisfaction measure(s) 08, all subordinate clinical units (children) will need to collect results for the patient satisfaction measure(s) on relevant patients. The subordinate clinics or child sites are still able to choose which of the unlocked other measures 08, if any, they want to incorporate in their clinical process.

As can be seen from the above, the present invention provides a user organization, such as a health care entity, a considerable amount of both flexibility and control in establishing and modifying the best practice goals for the organization. When a user at parent system 02 creates a child site, the user can specify the privileges that the administrator at that newly created site may have. To preserve this flexibility and control, the embodiments shown of the present invention do not allow the master set of measurements 14 to be deleted from the systems by any user other than the administrator at parent system 02 or the authorized users at a host web application provider site. Instead, the active 22 indicator 30 is used, in conjunction with the lock 26 indicator 30 to discontinue usage of a specific measurement 14, either temporarily or permanently.

Those skilled in the art will appreciate that other combinations of indicators 30 could be used without deviating from the spirit of the present invention, as could other approaches to authorization, privilege levels and editing or deletion.

Returning to FIG. 6, in the embodiments shown, parent system 02 can be implemented in several different ways. First, because it can be scaled from one to many different levels of users, parent system 02 can be implemented as a World Wide Web web-based application service, and the direct child systems C, might be diverse health care entities that subscribe to the system through parent system 02. In another embodiment, parent system 02 might be a health care insurer and its direct children might be health care entities. Then again, parent system 02, might be implemented at a primary health care provider site and the child sites might be affiliated clinics or practitioners. Or parent system 02 might be implemented at the office of a solo health care practitioner. Those skilled in the art will appreciate that different methods of implementation such as these and others are possible without deviating from the present invention.

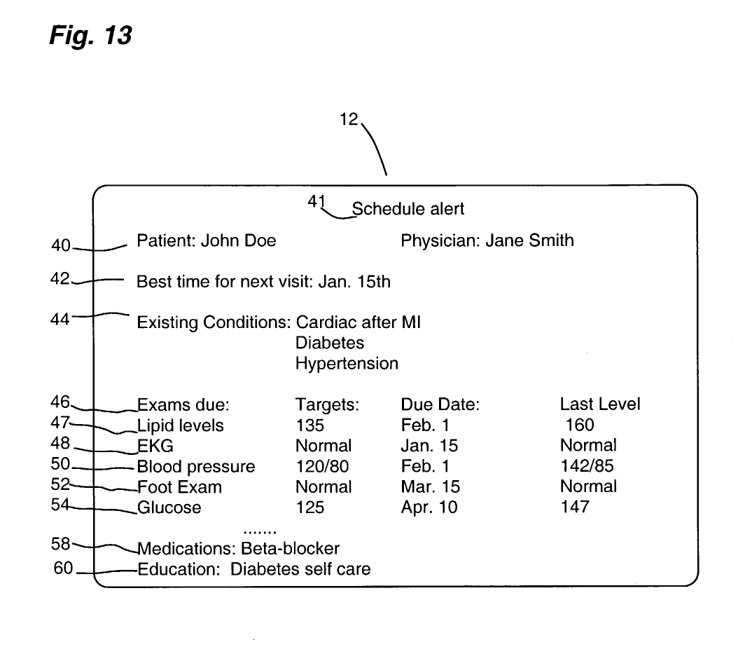

With reference now to FIG. 13, a sample report using the present invention is shown. On screen display 12, the present invention shows a schedule alert 41, for patient 40. In the embodiments shown, those of the master set of measurements 14 having a data value to be used for scheduling are used to select the nearest next scheduled visit date and provide an alert to the user. Since this patient is a cardiac patient after a myocardial infarction, who also has diabetes and hypertension, as indicated at line 44, the date values for measures 08 shown at lines 48-54 associated with either the cardiac status or the diabetes condition may have prompted this schedule alert. Note that at line 46, there is an indication of examinations that will be due at this visit, together with target values for those tests for the patient. As shown in FIG. 13, the present invention will find the earliest due date for all the measures 08 with due dates that the patient has. For example, measures 08 with two month, three month and four month intervals may be appropriate for a patient with several conditions. In this case, the invention would identify the earliest of these—a date two months from the last visit—as the time that an individual should return to the clinic. In the embodiments shown, the user can also specify the kinds of logic to be applied to conditions or measures. (See FIG. 14, for example, where goal logic is one of the items which can be defined for a measure 08.)

Still in FIG. 13, it can be seen that the term cholesterol has been changed for display purposes at line 47 to “lipid level.” In the embodiments shown, a user who is authorized to do so may rename the measure 08 for display purposes. This enables the system of the present invention to be customized to each user site's naming conventions, workflows and procedures. The present invention will treat the new name as an alias for the original name to insure that values can still be analyzed and compared properly based on the intrinsic meaning of the measure 08, not just the display name for the measure 08. So whether cholesterol is called cholesterol or lipid, the value will be treated as the cholesterol value in the database 04 for analytic purposes.

Again in FIG. 13, it can be seen that although two conditions 10, cardiac after MI and diabetes, call for a cholesterol test in the examples given above, the quick entry function of the present invention recognizes that a specific measure 08 has already been requested for the cardiac after MI condition 10 and quick entry does not duplicate it for diabetes. At the same time, the actual values returned when the visit occurs will be available for analysis in conjunction with both conditions 10, since the measure 08 for cholesterol is linked to both conditions 10.

In the embodiments shown, tests such as those called for by FIG. 13 may also be tracked by the system and further alerts given if a test is overdue. The present invention also allows a user to define a grace period for such a test. Thus, while January 15^{th} might be the best time to schedule the next visit for this patient, as indicated at line 42, this may be driven primarily by the cardiac after MI diagnosis. Users at this site may have defined a grace period as short as one month from the ideal date for this measure 08. For other measures 08, longer grace periods might be defined.

At line 58 of FIG. 13, the beta-blocker medication reminder for cardiac after MI is shown, as well, if this is part of the best practices adopted by this practitioner. In addition, at line 60, the system is indicating that education on diabetic self care is indicated. This recommendation may have been included as a one-time measure 08 for the initial diagnosis of diabetes as a condition 10. In the embodiments shown of the present invention, the system also defines which, if any, fulfillment activities—such as printing out the appropriate educational material for the patient at the time of the visit—need to be performed. Since the present invention provides a user with considerable flexibility in describing conditions 10 and measures 08, the embodiments shown make it possible for such fulfillment activities to be targeted as well. For example, if a patient has been newly diagnosed with diabetes a more general piece of educational material on self-care may be selected by the present invention for fulfillment purposes.

However, if a patient with previously diagnosed diabetes presents to the clinic with foot sores, an article on foot examination as it relates to diabetes can be defined as a fulfillment activity for this patient, based on the conditions 10 and measures 08.

Thus, as seen in FIG. 13, the embodiments shown of the present invention enable a health care entity to establish practice guidelines for the care and examination of patients using evidence-based medicine applied in a practical manner. In this example, the system provides an alert that patient John Doe is due for a visit at which several key tests should be done, and his recommended medication for existing conditions is shown as well.

This information is supplied by the system to the users at the site who need to know the information, such as the receptionist who schedules visits as well as the appropriate health care providers. When the patient comes in for the visit, the health care practitioner can be given a printed form containing the information, if he or she so desires, or the practitioner can use screen displays on a computer or terminal or personal digital assistant or similar device to conduct the visit. The present invention enables the health care entity to provide outreach to patients, to insure they are actually scheduled for visits, and to remind the practitioner not only what the patient's existing health status is, but which tests and measures can best be used to improve that status, according to the known, evidence based data.

In addition, as shown by FIG. 13, the actual results for each patient against these goals can be tracked as well, not only at the local site level, but throughout the system. This, in turn, means that the health care entity has much more useful information with which to evaluate its overall efficacy and efficiency.

In addition to enabling the health care entity to improve the quality of care, the system may also make it possible for the health care entity to lower rates for malpractice insurance by showing, for example, that evidence based goals and tests from the most respected studies are scheduled and conducted in a timely manner. The ability to demonstrate this to insurers may also improve revenue collection for the provider if the plan or other sponsoring entity is rewarding quality of care.

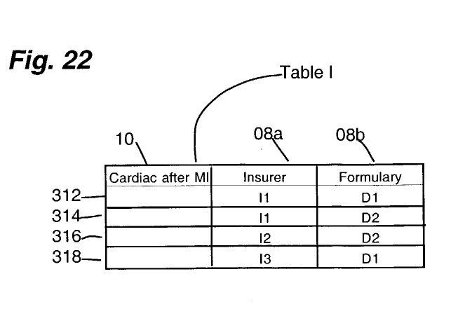

Similarly, and turning briefly to FIG. 22, the present invention can also be used to assist in implementing prescription formulary goals of health care insurers. Many heath care insurers have a list of approved prescription drugs for which they will provide insurance coverage. These lists can differ significantly from one insurer to the next, however, because the insurer may be providing coverage for a particular medication based on a set of underlying assumptions, which differ from other insurers. For example, a company which offers health insurance at a particular rate for a population that is primarily composed of the elderly makes certain assumptions about compensation from other sources, such as medicare. A company offering health plans to employees of industries known to employ a typically younger, healthier population may set its rates quite differently. The formulary for one insurer may not include prescription drugs for which medicare coverage is not available. Another insurer may not include coverage for newer prescription drugs, because its rates have been set based on a population less likely to need them. An insurer may also provide coverage for several different drugs for the same condition, but provide co-pay incentives to use one more than others. In FIG. 22, for example, Table I shows how the present invention can be used to accommodate this. For a condition 10 such as cardiac after myocardial infarction, there may be two (or more) prescription beta-blockers—D1 and D2 in FIG. 22—which can be prescribed for it. D1 may be a standard beta-blocker which has been around for years, and which can safely be prescribed for everyone. D2, however, may be a more recently developed beta-blocker which is also safe for everyone, but is especially useful for reducing known side effects likely to occur when a patient is taking other medications for other conditions.

In the example of FIG. 22, insurer I1, may have two different co-pay structures. For beta-blocker D1, the older drug, it may have a more generous co-pay feature, say a $5 co-payment. However, for D2, insurer I1, may wish to discourage general use of the newer, more expensive drug by patients who do not have the other complicating conditions, by having a co-payment of $30 for it for those patients. The present invention would enable insurers and health care providers to structure goals for formularies which would encourage a health care practitioner to prescribe the appropriate drug for a patient covered by a particular insurer. This can be seen in FIG. 22, in which, for the condition 10 cardiac after MI, insurer I1 provides two formularies, one for drug D1, which may be the general purpose beta-blocker and one for D2, the newer one designed to minimize side effects when used with other prescriptions. In this example, a condition 10 is linked with two tiers of measures 08—here 08a, which insurer and 08b which drug is in the formulary. Similarly, the goal logic for the measure 08 associated with the more expensive drug D2, could make it clear that the co-pay for drug D2 is only $5.00 when the patient takes more than some specified number of other drugs, such as four, but the co-pay will be $30.00 for patients who take fewer than four other drugs. Thus, the system can be used to set the goals for a medical needs driven co-pay structure.

Thus, the present invention enables a health care entity such as a hospital or clinic working with patients who have coverage from different insurers to define a set of best practices which will prescribe the best drug for a specific patient, given that patient's present health status, insurance coverage and personal preferences. The system also makes it very easy to analyze and report on how well the target best practice goals are being met.

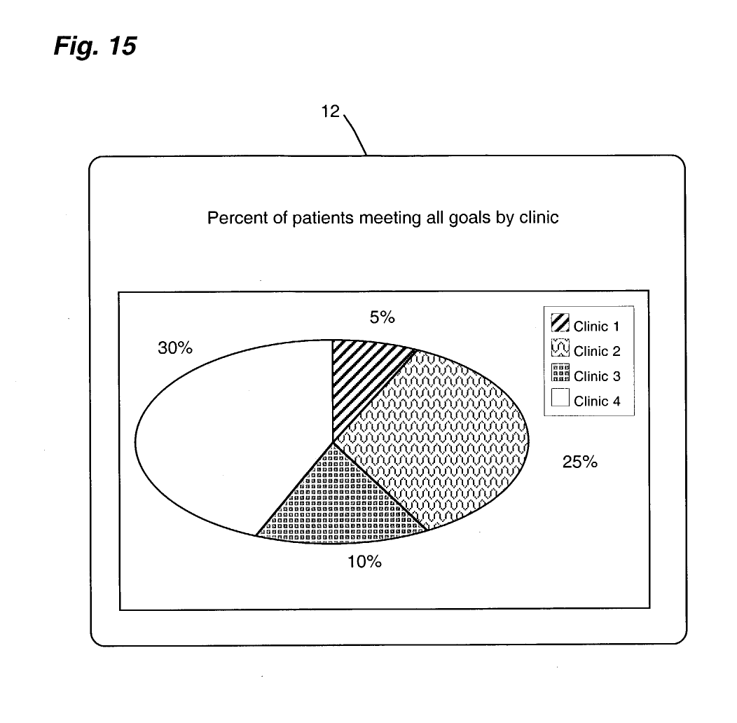

In FIG. 15, for example, screen display 12 shows a pie chart that can be produced by the system indicating the percentage of patients meeting all goals. In this example, there are four clinics, and clinics 2 and 4 have a much higher percentage of patients meeting all target goals—25% and 30% respectively. Since the embodiment shown of present invention is also a longitudinal system, that is, one that is capable of tracking patient or subject results over longer periods of time as well, this kind of information can be studied as new goals and measures are introduced to see how well they work.

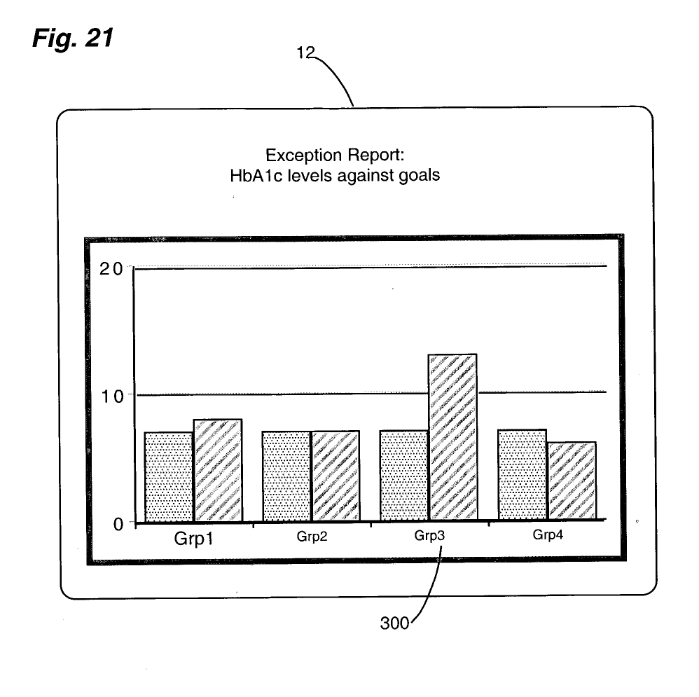

With reference now to FIG. 21, it is also possible to use the present invention to identify problems or exceptions or patients that might need special attention. In FIG. 21, a bar graph report is shown for several group child sites Grp1 through Grp4 in a provider entity. The measure being evaluated is HbA1c levels against goals. For diabetic patients, studies have shown that this measure of glycosolated hemoglobin (the amount of glucose that is irreversibly bound to hemoglobin in the blood) is directly related to the concentration of glucose in the blood. Red blood cells have a life span of approximately 90 days. Consequently, measuring the amount of glycosolated hemoglobin (HbA1c) gives a good indication of the average blood sugar control in the patient during the 60 to 90 days preceding the test. A value of 7 or lower for this is normal or better than normal. A value over 8.5 is considered elevated, and values over 12 are considered seriously elevated.

As can be seen in FIG. 21, the patients in Grp3 300 have measures that seem to average above 10. This might serve as an alert for the administrators at Grp3 300 to discuss this situation with the providers in Grp3 300, who may not be aware of the situation. Of course, it is possible that everyone knows about this because the providers there might be specialists who handle the more complex diabetic cases. However, if that is not true, then the present invention may have helped to identify a problem where more education for both patients and providers can be useful.

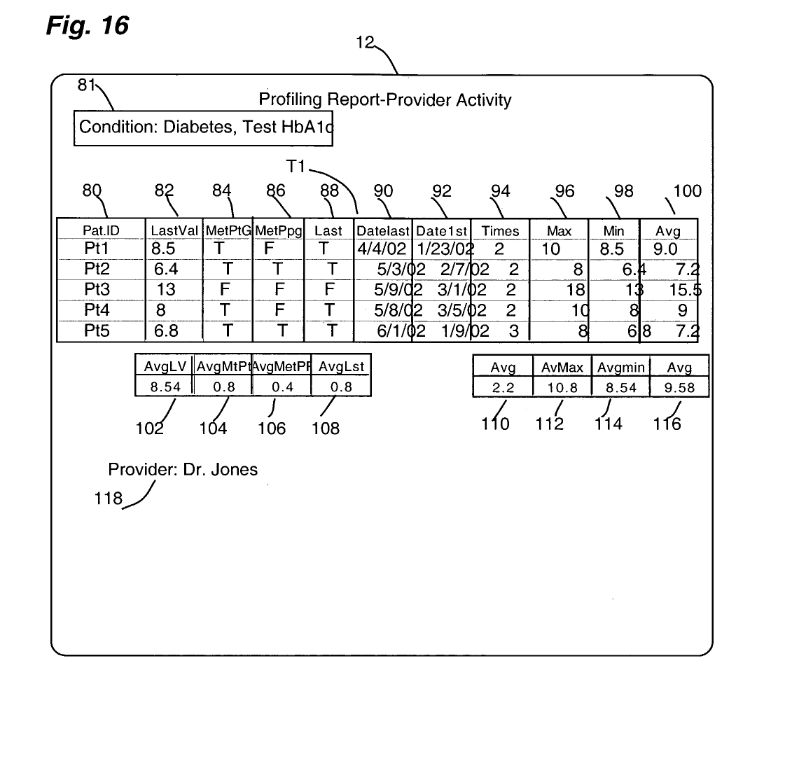

In a similar vein, but turning now to FIG. 16, a provider activity report for diabetic patients of a provider is shown in screen display 12. As seen in header 81, the condition 10 for which a report has been requested is diabetes and the measure 08, requested for it is HbA1c values. In this report, a summary of the values for measure 08 for all patients with diabetes who are under the care of provider 118, are shown. In this report, there is a patient id 80 column, a last value 82 column, a met patient goal 84 column, a met population goal 86 column, and a value for the last 88 measure 08 column. In addition, the date of the last 90 and the first 92 tests for this measure 08 are shown, as are the number of times 94 the patient has been tested, the maximum 96 test values for this patient, the minimum 98 value for this patient, and the average 100 value for this patient. Averages of these for all patients are included at items 102-116. In this embodiment, the provider has set (presumably with the patient) personal patient goals, as well as using the overall population goals for this measure 08. Patient PT1 for example, has met his or her personal goal in the most recent test, even though he or she has not met the overall population goal. The flexibility the present invention provides to set and track these kinds of measures makes it much easier for the provider to see quickly which patients are making real progress and which might be falling behind.

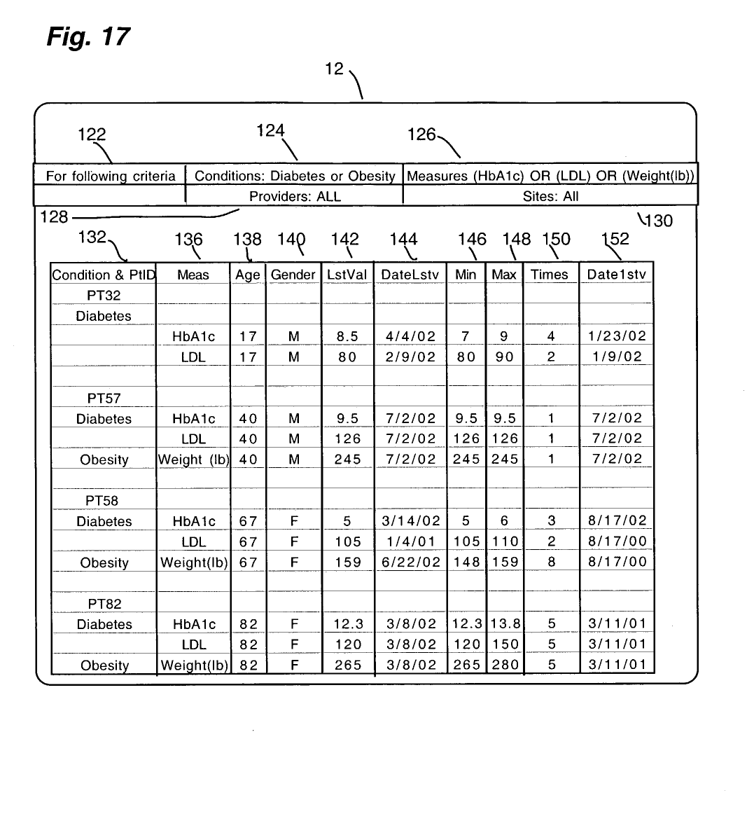

Similarly, in FIG. 17, a detailed exception report on all patients with diabetes or obesity with all providers in the system is shown, with three key measures included: HbA1c, LDL and weight. Age 138 and gender 140 information is included as well. This report could be refined by requesting an exception report only showing those patients with diabetes or obesity who have not met the population goals for these measures 08. In addition, this information can be provided in the appropriate data format to analysis programs for further study. Thus, if patients with one group within the overall health care entity are doing significantly better than others, it might be feasible to investigate what the providers at that group are doing to produce such good results. The group with the improved performance may have set their goals to different values from those used by others. If it turns out these goals work more effectively in other test groups as well, the administrator for the whole entity can easily propagate these new goals to all or make them available to all in the system using the present invention's setting and scaling features.

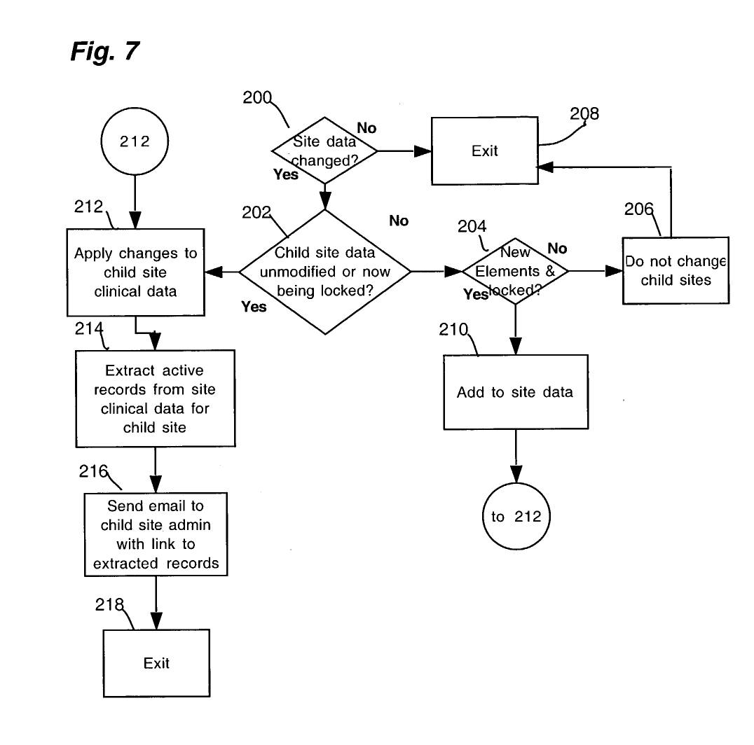

These setting and scaling features are illustrated in flow diagram form in FIG. 7. When an administrator at a parent site makes a change to that site's clinical data 19, the invention at decision block 200 checks to see if a change has been requested, and if so, it next checks at decision block 202 to see if the child site clinical data 19 for that parent is still unmodified or is now being locked. If either of these is true, then the invention, at step 212 applies the changes to the child site's clinical data 19 in database 04. Next, at step 214, the setting function 03 of the present invention extracts from database 04 the active records from site clinical data 19 for the child site, and then, at step 216, sends an e-mail or similar notification to the child site administrator with a link to the extracted records and then exits at step 218. If the child site clinical data 19 has already been modified, however, the invention at step 204 checks to see if this change is adding new elements and locking them. If it is, at step 210 they are added to the site clinical data 19 and processing proceeds as described at step 212. If the modifications are not new elements which are being locked, the child site data is not changed.

If changes have occurred and an e-mail has been sent, in the embodiments shown the administrator at the child site can use the link contained in the e-mail (and a one-time userid and password also contained in it) to log on to a secure site that automatically downloads the site clinical data 19 to the child site and replaces the child sites' existing site clinical data 19 file. As can be seen, the site clinical data 19 files of the present invention contain the goal or practice values, not the actual results values for individual patients. Thus, they can be changed and modified, as appropriate, without losing any patient results data using the present invention.

Those skilled in the art will appreciate that while the embodiments shown use distributed databases and web based technologies, the present invention could also be implemented with a single centralized database, or for a single site.

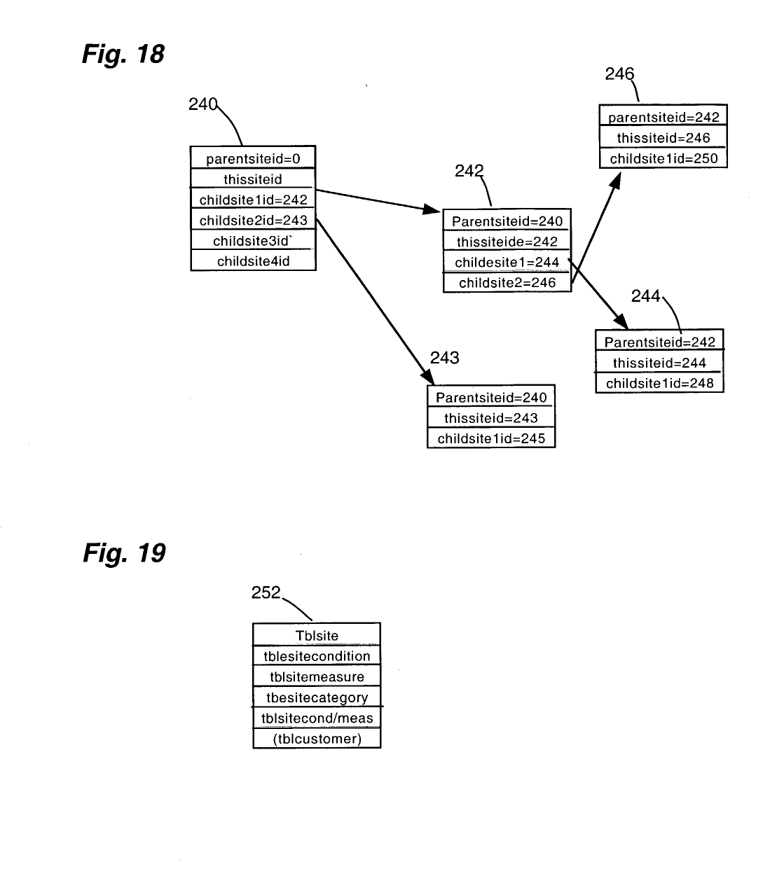

The process described in FIG. 7 is repeated for all children, grandchildren, etc. of the parent site. Setting function 03 finds the children by using a parent identifier (id) field. It searches for all sites that have a parent id field equal to the current sites' site id field. By keeping track of all site id's at each level, the system can find all children, no matter how many levels down they are. This can be seen by looking briefly at FIG. 18, which shows site tables for a parent site 240, two of its children sites 242 and 243 and two grandchildren sites 246 and 244.

Referring briefly to FIG. 19, the embodiments shown use a series of tables 252 to store the site identifiers, conditions 10, measures 08, categories 11, linked conditions 10 and measures 08, and so on. Those skilled in the art will appreciate that different techniques for organizing the data can be used without deviating from the spirit of the present invention. For example, tree structures, hierarchical databases, relational databases and so on could be used without deviating from the present invention.

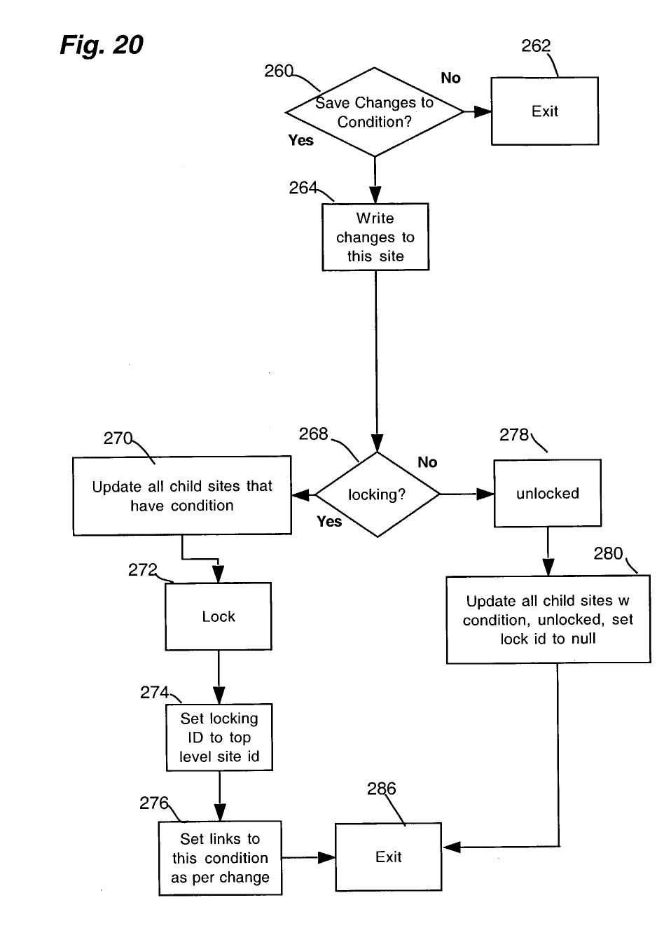

With reference now to FIG. 20, a more detailed flow diagram is provided to illustrate how setting function 03 of the present invention handles changes in conditions 10 at a site. As seen there, at step 260, setting function 03 checks to see if a “save changes” request has been made. If not, it exits at step 262. If the request has come in from a screen display 12, the invention, at step 264 writes the changes for this site. Next, at step 268 setting function 268 checks to see if this condition is being locked. If it is, at step 270, the invention will update all subordinate sites that have the condition, and at step 272 it will lock the condition. At step 274 it sets the locking id to that of the top level site id. Finally, at step 276, it will set any links to this condition to correspond to this change, before exiting at step 286. If, however, as determined at decision block 278, the lock flag for this condition 10 has been turned off, the invention updates all subordinated sites with this condition 10 to unlocked, and sets the lock id to null at step 280. Then the system exits at 286.

The embodiments shown are implemented as a web based application service using Microsoft corporation's ASP, ASP.Net and Visual Basic.Net development tools. However, those skilled in the art appreciate that other computer platforms such as SUN Microsystems workstations, or IBM Mainframes could be used as well, either in client server or centralized or distributed network configurations. While the embodiments shown are programmed using the above mentioned development tools, other programming languages and screen display languages can be used as well. Similarly, while software has been used along with the above computer systems to implement the invention, part or all of the invention could be included in firmware or hardware circuitry without deviating from the spirit of the present invention.

What is claimed is:

1. A system for flexibly setting measurements in a tracking system having the ability to track data about a subject, comprising:
a stored database containing global data and site specific data;
a master set of measurements stored in the global data, such measurements being propagated to the site specific data, as requested by a user;
an indicator associated with each measurement which indicates what may be changed;
a setting function that enables a user to change site specific data in accordance with the indicators associated with the measurements, so that the measurements for that site, including any changed by the user, serve as the goals for any schedules, examinations, and tests for evaluating results for subjects being tracked at that site.

2. The apparatus of claim 1, in which the results for subjects being tracked at that site further comprise a plurality of results collected over time for subjects being tracked at that site.

3. The apparatus of claim 1, wherein the master set of measurements further comprises a condition which is potentially applicable to a subject being tracked.

4. The apparatus of claim 1, wherein the master set of measurements further comprises a measure which is potentially applicable to a subject being tracked.

5. The apparatus of claim 1 wherein the master set of measurements further comprises a master set of measurements which is shared amongst a plurality of users.

6. The apparatus of claim 1, wherein the master set of measurements further comprises a set of best practices for use by a user.

7. The apparatus of claim 1, wherein the master set of measurements further comprises evidence based health care practices for use by health care entity users.

8. The apparatus of claim 1, wherein the master set of measurements further comprises pharmaceutical formularies provided by insurers for use by health care providers.

9. The apparatus of claim 1, wherein the master set of measurements further comprises medical needs driven co-pay structure best practices for use by a user.

10. The apparatus of claim 1, wherein the site specific data stored in the database further comprises data which applies to sites which are subordinated in familial relationships such as child site subordinated to a parent site.

11. The apparatus of claim 10, wherein the setting function further comprises a function which can be used to change site specific data at a subordinated site.

12. The apparatus of claim 1, wherein the site specific data stored in the database further comprises data which can be scalably propagated to a plurality of sites.

13. The apparatus of claim 1, wherein the master set of measurements further comprises measurements described by categories which control the display of the master set of measurements.

14. The apparatus of claim 1, wherein the master set of measurements further comprises measurements described by answer types which control the way in which the values for the measurements are stored and analyzed.

15. The apparatus of claim 10, wherein the indicators further comprise an active indicator, which, if set to true at a parent site, allows a measurement to be used at all sites subordinated to that parent site, and if set to false at a parent site, prohibits the use of that measurement at that parent site but permits its use at all sites subordinated to that parent.

16. The apparatus of claim 1, wherein the indicators further comprise an approve indicator, which, if set to true, allows the measurement to used by all sites.

17. The apparatus of claim 10, wherein the indicators further comprise a lock indicator, which, if set to true at a parent site requires that all subordinated sites in the familial relationship of that parent site must use the measurement unchanged.

18. The apparatus of claim 1, wherein the setting function further comprises a schedule planner which analyzes the measurements to determine appropriate schedules for evaluations and tests, if any, of a subject.

19. The apparatus of claim 18, wherein the schedule planner further comprises a quick entry function which eliminates any duplicative evaluations and tests which might be indicated for a subject.

20. The apparatus of claim 18, wherein the schedule planner further comprises a grace period planner which enables a user to specify a grace period for scheduled tests and evaluations.

21. The apparatus of claim 10, wherein the tracking system further comprises a scalable tracking function which can track results against measurement goals at any site level in a familial relationship.

22. A method for flexibly setting measurements in a tracking system having the ability to track data about a subject, comprising the steps of:
storing global data and site specific data in a database;
storing a master set of measurements in the global data, such measurements being propagated to the site specific data, as requested by a user;
associating an indicator with each measurement which indicates what may be changed;
setting site specific data changes made by a user in accordance with the indicators associated with the measurements, so that the measurements for that site, including any changed by the user, serve as the goals for any schedules, examinations, and tests for evaluating results for subjects being tracked at that site.

23. The method of claim 22, in which the step of setting site specific data changes further comprises the step of collecting a plurality of results over time for subjects being tracked at that site.

24. The method of claim 22, wherein the step of storing a master set of measurements further comprises the step of storing a condition which is potentially applicable to a subject being tracked.

25. The method of claim 22, wherein the step of storing a master set of measurements further comprises the step of storing a measure which is potentially applicable to a subject being tracked.

26. The method of claim 22 wherein the step of storing a master set of measurements further comprises the step of storing a master set of measurements which is shared amongst a plurality of users.

27. The method of claim 22, wherein the step of storing a master set of measurements further comprises the step of storing a set of best practices for use by a user.

28. The method of claim 22, wherein the step of storing a master set of measurements further comprises the step of storing evidence based healthcare practices for use by health care entity users.

29. The method of claim 22, wherein the step of storing a master set of measurements further comprises the step of storing pharmaceutical formularies provided by insurers for use by health care providers.

30. The method of claim 22, wherein the step of storing a master set of measurements further comprises the step of storing medical needs driven co-pay structure best practices for use by a user.

31. The method of claim 22, wherein the step of storing site specific data in the database further comprises the step of storing data which applies to sites which are subordinated in familial relationships such as child site subordinated to a parent site.

32. The method of claim 31 wherein the step of setting site specific data changes further comprises the step of setting site specific data changes at a subordinated site.

33. The method of claim 22, wherein the step of storing site specific data in the database further comprises the step of storing data which can be scalably propagated to a plurality of sites.

34. The method of claim 22, wherein the step of storing a master set of measurements further comprises the step of storing measurements described by categories which control the display of the master set of measurements.

35. The method of claim 22, wherein the step of storing a master set of measurements further comprises the step of storing measurements described by answer types which control the way in which the values for the measurements are stored and analyzed.

36. The method of claim 31, wherein the step of associating indicators further comprises the step of associating an active indicator, which, if set to true at a parent site, allows a measurement to be used at all sites subordinated to that parent site, and if set to false at a parent site, prohibits the use of that measurement at that parent site but permits its use at all sites subordinated to that parent.

37. The method of claim 31, wherein the step of associating indicators further comprises the step of associating an approve indicator, which, if set to true, allows the measurement to used by all sites.

38. The method of claim 31, wherein the step of associating indicators further comprises the step of associating a lock indicator, which, if set to true at a parent site requires that all subordinated sites in the familial relationship of that parent site must use the measurement unchanged.

39. The method of claim 22, wherein the step of setting site specific data changes further comprises the step of analyzing the measurements using a schedule planner to determine appropriate schedules for evaluations and tests, if any, of a subject.

40. The method of claim 39, wherein the step of analyzing the measurements using a schedule planner further comprises the step of using a quick entry function to eliminate any duplicative evaluations and tests which might be indicated for a subject.

41. The method of claim 39, wherein the step of analyzing the measurements using a schedule planner further comprises the step of using a grace period planner to enable a user to specify a grace period for scheduled tests and evaluations.

42. The method of claim 31, wherein the step of setting site specific data changes further comprises the step of applying a scalable tracking function for tracking results against measurement goals at any site level in a familial relationship.

43. A machine readable medium for storing a system for flexibly setting measurements in a tracking system having the ability to track data about a subject, the system including a stored database containing global data and site specific data; a master set of measurements stored in the global data, such measurements being propagated to the site specific data, as requested by a user; an indicator associated with each measurement which indicates what may be changed; and a setting function that enables a user to change site specific data in accordance with the indicators associated with the measurements, so that the measurements for that site, including any changed by the user, serve as the goals for any schedules, examinations, and tests for evaluating results for subjects being tracked at that site.
